(19) United States
(12) Patent Application Publication
(10) Pub. No.: US 2006/0015007 A1
(43) Pub. Date: Jan. 19, 2006

(54) Resectoscope comprisig positioned optics

(75) Inventors: Thomas Aue, Hamburg (DE); Werner Buaa, Reinbek (DE); Pieter Brommersma, Bargteheide (DE); Felix Nussbaum, Hamburg (DE)

Correspondence Address: RANKIN, HILL, PORTER & CLARK LLP, 4080 ERIE STREET, WILLOUGHBY, OH, 44094-7836, US

(73) Assignee: OLYMPUS WINTER & IBE GMBH, Hamburg (DE)

(21) Appl. No.: 10/531,838

(22) PCT Filed: Oct. 2, 2003
(86) PCT No.: PCT/EP03/10951

§ 371 Date: Apr. 18, 2005

(30) Foreign Application Priority Data
Oct. 19, 2002 (DE) 102 48 836.3

(51) Int. Cl. A61B 1/00

(52) U.S. Cl. 600/105

(57) ABSTRACT

A urological resectoscope is disclosed, having a tubular shaft (1), through which an optical system (2) and a longitudinally moveable instrument carrier (5) pass. The optical system (2) is supported with respect to the tubular shaft (1) at a spacing from the proximal end of the tubular shaft (1) with a support device (9, 12). The carrier (5) is positioned with a radial positioning device (10, 15) and a circumferential angular positioning device (11, 12). The support device (12, 18, 18′) is so arranged adjacent to the optical system (2) in the region in which the carrier (5) extends, that it supports the optical system (2) with respect to the tubular shaft (1) with a two point support system and imparts lateral guidance to the carrier (5) in the circumferential angular direction.

### BACKGROUND OF THE INVENTION

1. Field of the Invention

The invention relates to a urological resectoscope of the type having a tubular shaft through which an optical system and a longitudinally moveable instrument carrier pass.

2. Description of Related Art

Resectoscopes constitute the principal device in urological surgery and are used, in particular, for prostate resections. They may also be used in gynaecology. As the instrument they have a knife or generally the conventional cutting loop, to which high frequency may be applied, which may be advanced forwardly beyond the tubular shaft. The instrument is used in the region in front of the distal end of the tubular shaft whilst optically viewing through the optical system. The tubular shaft can be a simple tube or can consist of an outer shaft and an inner shaft, when the resectoscope is constructed in the form of a continuous flow resectoscope, the optical system and the elongate instrument carrier carrying the instrument at its distal end being arranged within the inner shaft.

Provided at the proximal end of the potentially multi-tube shaft, which may commonly may be decoupled, is an operating device, starting from which the optical system, which is commonly replaceable, may be slid into the system, and which is engaged by the hand of the user. A sliding carriage is also provided there in the conventional construction, to which the proximal end of the instrument carrier is fastened in order that it may be longitudinally moved and to which current may be applied, if it is a high frequency operated instrument.

Precise positioning of the instrument in the radial and rotational direction is of importance in resectoscopes for a precise mode of operation. Furthermore, the optical system must be held in a predetermined position in order to ensure a reproducible viewing angle.

The known construction of resectoscopes of the type referred to above, which is currently common in the art, is illustrated in FIG. 1.

Only the distal end region of the shaft is shown with a tubular shaft 1 and an optical system 2 with an objective 3 and with an optical guide tube 4, which guides the optical system over a proportion of the length of the tubular shaft 1. The optical guide tube 4 extends, in the conventional construction, through the main body, not shown, of the resectoscope, to which the tubular shaft 1 may be coupled, and extends through the operating device and commonly through the sliding carriage provided there.

An instrument carrier 5 (referred to below as the carrier), which is constructed in the form of an elongate rod, extends at its proximal end to the sliding carriage, which is not shown, with which it is connected and in electrical contact. It extends straight through the tubular shaft 1 to a branch point 6, at which it branches to form a fork 7, the two branches of which extend laterally around the optical system 2 and carry the cutting loop 8, to which high frequency may be applied, at their ends, instead of which a knife can also be provided as an instrument in another embodiment.

In the conventional construction of known resectoscopes, the illustrated tubular shaft 1 is the inner shaft of a shaft system with two concentric shafts. Also provided on the shafts in the distal end region is an annular insulating body. Flushing liquid is conducted through proximally disposed flushing connections which are not shown, to the interior of the tubular shaft 1 and is drained away by suction through the space between the two tubular shafts.

With regard to the positioning problem outlined above, the known prior art provides the following as shown in FIG. 1:

The optical guide tube 4 is supported with respect to the tubular shaft 1 with a nose 9, which is connected in the exemplary construction, to a guide tube 11, which for its part is connected to the optical guide tube 4.

The exact positioning of the instrument, that is to say of the cutting loop 8, is effected (with polar co-ordinates seen in the cross section of the tubular shaft 1) by a radial positioning device and a circumferential angular positioning device, which are separately constructed in the prior art. Secured to the carrier 5 is a sliding tube 10 constituting the radial positioning device, which is slideably guided on the optical system 2 and thus ensures the radial distance between the precisely positioned optical system 2 and the carrier 5.

The guide tube 11, through which the carrier 5 passes, serves as the circumferential angular positioning device and holds it at a fixed circumferential angle.

This known construction has disadvantages, particularly as regards the guide tube 11, which is difficult to clean as a result of its small diameter. Furthermore, the optical guide tube 4 is absolutely necessary for the mounting of the guide tube 11 and the nose 9, which positions the optical system. As a result of the optical guide tube 4, the overall construction of the resectoscope is made more expensive and cleaning problems are produced as regards also the narrow guide tube 11.

DE 19631677 C1 discloses an endoscope of a type completely different to that referred to above, which serves to sever perforated veins when removing varicose veins in the leg. Disposed in a tubular shaft of very large diameter is an optical system, which is supported by means of a ramp in order to secure its position. As a result of the particular features of this special construction, no suggestion can be derived for the construction of resectoscopes.

### SUMMARY OF THE INVENTION

The object of the present invention resides in simplifying a resectoscope of the type referred to above structurally and as regards the cleaning problems.

In accordance with the invention, the optical system is supported in the angular range of the carrier with respect to the tubular shaft with a two-point support system, whereby it is reliably positioned. The support can advantageously be effected such that the optical system is pressed against the tubular shaft at a third, opposite position so that it is situated in a precise three-point positioning system. The support device imparts lateral guidance to the carrier in order to prevent variations in the circumferential angular direction. For its radial positioning, the carrier can be guided in the conventional manner, for instance as illustrated in FIG. 1, with the sliding tube 10 on the optical system 2. The guide tube 11 is omitted in the construction in accordance with the invention with its cleaning problems. The entire optical guide tube can also be saved. The possibility is also produced by comparison with the prior art, as is illustrated in FIG. 1, of arranging the entire positioning device of the optical system and the carrier closer to the distal end of the tubular shaft and thus with a better positioning action on the instrument itself.

Two fixed angular positioning webs in lateral sliding contact with the carrier produce a good circumferential angular positioning of the carrier and thus of the instrument and a reliable two point positioning of the optical system with respect to the tubular shaft.

The webs can be constructed in the interior of the tubular shaft on the latter, e.g. by soldering or by the formation of indentations on the tubular shaft. A very simple and precisely positioning construction is thus produced.

As already mentioned, the optical guide tube can be saved in accordance with the invention. If, however, an optical guide tube is present for other reasons, then the webs are fastened to the optical guide tube and support the latter, and thus the optical system, with respect to the tubular shaft. This construction has the advantage that a smooth, conventional tubular shaft may be used.

As already mentioned, the webs serve to guide the carrier in the circumferential direction. For the purpose of radially guiding the carrier, it can be guided on the optical system in the known manner with the sliding tube. Advantageously, however, the webs are so constructed that they guide the carrier in its radial position also with guide profiles. The sliding tube on the carrier can then be omitted.

The carrier can be guided directly on the webs in its rod-shaped construction. Preferably, however, strips are provided on the carrier in the length region, in which it comes into contact with the webs, which ensure sliding contact with the webs. Particularly precise sliding contact can be ensured with the strips, particularly if a profiled engagement is necessary.

As already mentioned, the support device can be provided connected to the tubular shaft e.g. in the form of webs, or alternatively connected to the optical guide tube, if one is present. Alternatively, the support device is connected to the carrier. Changes to the resectoscope are thus superfluous. The support device again supports the optical system with respect to the tubular shaft and imparts guidance to the carrier in the peripheral angular direction. In the event of longitudinal movement of the carrier, it slides on the optical system and the tubular shaft whilst maintaining the guidance.

The non-circular peripheral profile of the tubular shaft results in a precise angular positioning of the support device sliding in profiled engagement with the tubular shaft and thus in a better circumferential angular guidance of the carrier.

### BRIEF DESCRIPTION OF THE DRAWINGS

FIG. 1 shows the known construction of resectoscopes which is currently common in the art,

FIG. 2 is a longitudinal sectional view corresponding to FIG. 1 of a resectoscope in accordance with the invention,

FIGS. 3-5 are sectional views on the line 3-3 in FIG. 2 of different embodiments,

FIG. 6 is a sectional view corresponding to FIG. 2 of a further embodiment,

FIG. 7 is a sectional view on the line 7-7 in FIG. 6 and

FIG. 8 is a view corresponding to FIG. 7 of a further embodiment.

### DETAILED DESCRIPTION OF THE INVENTION

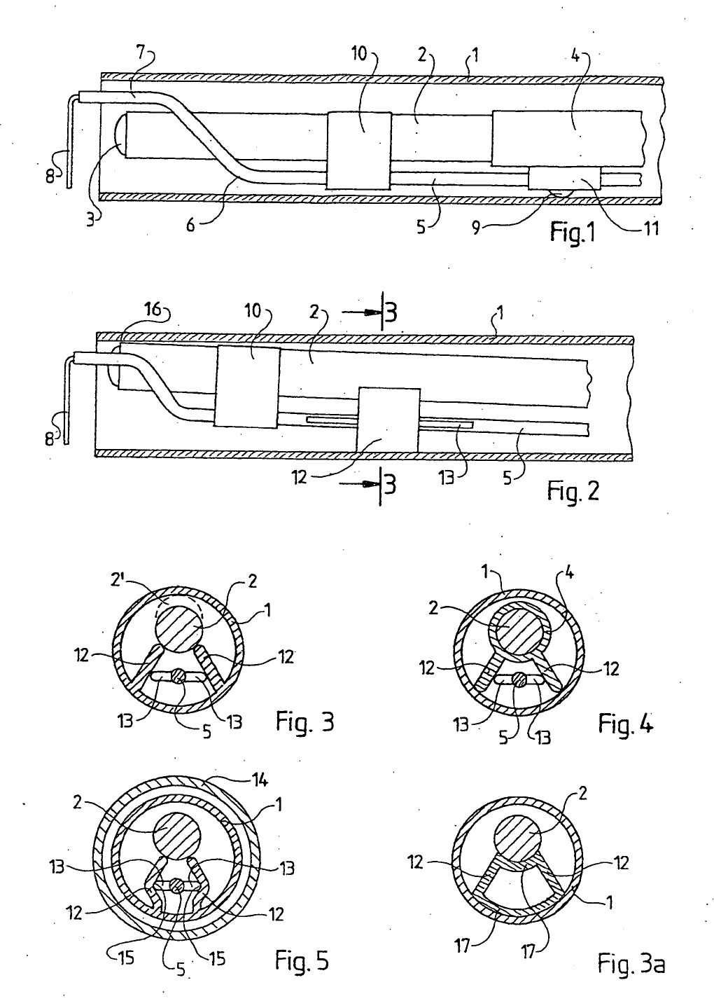

FIGS. 2 and 3 show a first embodiment of the invention corresponding to the extent possible with FIG. 1 and using, to the extent possible, the same reference numerals. The tubular shaft 1 or the inner shaft of a multi-tube resectoscope is again shown. The carrier 5, which substantially corresponds to that in FIG. 1, is guided in the same manner in this embodiment at a radial spacing on the optical system 2 with the sliding tube 10, as in the known construction of FIG. 1.

The construction of FIG. 2 has no optical guide tube. The support of the optical system 2 with respect to the tubular shaft 1 is effected by means of two webs 12, as is shown in the sectional view on the line 3-3 in FIG. 3. The webs extend in the radial direction between the optical system 2 and the tubular shaft 1 and are constructed integrally with the tubular shaft 1 in the exemplary embodiment of FIGS. 2 and 3. As shown in FIG. 3, a reliably positioning engagement for the optical system 2 is produced. The webs 12 can also be soldered to the tubular shaft or formed on it in the form of indentations.

As shown in FIG. 3, the carrier 5 extends between the webs 12 and is positioned by it in the circumferential angular direction. For this purpose, the carrier 5 has lateral strips 13 in its longitudinal displacement region, with which is engages the two webs 12.

FIG. 3a shows a structural variation to FIG. 3, in which the two webs 12 are connected to inner and outer shells 17 to form a tubular plate body, which is closed in the cross section of FIG. 3a and is secured to the tubular shaft 1 by soldering or in some other manner and which, as may be seen, fulfils the same supporting function as shown in FIG. 3. The carrier 5, which is not shown in FIG. 3a, can be laterally supported in the same manner as shown in FIG. 3.

FIG. 4 shows a structural modification to the embodiment to FIGS. 2 and 3, in which the optical guide tube 4, which may be seen in FIG. 1, is provided. The webs 12 are secured in this case, as shown in FIG. 4, to the optical guide tube 4 and engage the tubular shaft 1 in a reliably supportive manner. The carrier 5 is supported in the peripheral direction between the webs 12, as shown in FIG. 3. The tubular shaft 1 can be constructed in this case as a continuously smooth tube.

If one compares the embodiments of FIG. 3 and FIG. 4, it will be seen in a further comparison with FIG. 1 that in the embodiment of FIG. 3 there is not only the advantage that no optical guide tube 4 is necessary but also the advantage that the circumferential angular positioning of the carrier 5 can be effected not on the optical guide tube, that is to say relatively remote from the distal end of the tubular shaft 1, but further forwards in the region of the sliding tube 10, that is to say with a better positioning effect on the cutting blade 8.

FIG. 5 shows a further alternative embodiment, whereby it is additionally illustrated in this case for explanatory purposes that the tubular shaft 1 can be an inner shaft, which is surrounded by an outer shaft 14.

FIG. 5 again shows webs 12, which are connected to the tubular shaft in accordance with the embodiment of FIG. 3 and supportingly engage the optical system 2. However, in the embodiment of FIG. 5, the webs are of kinked shape with an angular profile 15 extending in the longitudinal direction of the shaft, in which the strips 13 of the carrier are subjected to precise guidance, which reliably guides the carrier 5 not only in the circumferential angular position but also in the radial spacing from the optical system 2. The sliding tube 10 illustrated in FIGS. 1 and 2 can be omitted in this construction.

In the embodiments illustrated in FIGS. 3 to 5, the carrier 5 is positioned against the webs 12 with strips 3. The webs 12 can, however, can also be positioned closer together so that they exactly guide the carrier 5 directly, that is to say without strips 13.

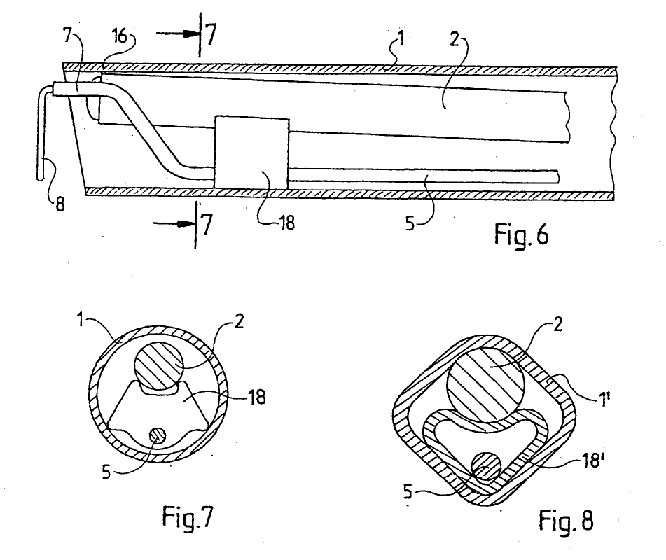

In the illustrated embodiments, the optical system is positioned against the webs 12 with a two point engagement with bending stressing of the optical system or, as shown in FIG. 4, with stressing also of the optical guide tube 4. It can be held by the webs 12 against the opposite inner surface of the tubular shaft 1, as illustrated in FIGS. 2 and 6 with the contact point 16 and as indicated in FIG. 3 with 2′. There is then a precisely positioning three point engagement. FIGS. 6 and 7 show another embodiment, in which, instead of the previously described webs 12, a support device 18 is provided which is shown in the form of a block with a profiled periphery, which is secured to the carrier 5 and moves in sliding contact with the optical system 2 and the inner surface of the tubular shaft 1 with longitudinal movement of the carrier 5. As shown in FIG. 7, the support device 18 is in two point engagement with the optical system 1 and also in two point engagement with the tubular shaft 1 and basically imparts to the optical system 2 the same two point support with respect to the tubular shaft 1, as is illustrated in e.g. FIG. 3. It also ensures that the carrier 5 has good lateral support and, as shown in FIG. 7 and indicated with the contact point 16 in FIG. 6, presses the optical system 1 at a third engagement point against the tubular shaft 1 to produce a reliable three point support system.

As a modification of FIG. 7, FIG. 8 shows a structural variant, in which the tubular shaft 1′ is of non-circular profile in the illustrated manner. The support device 18′ is constructed in the form of a tubular body with a profiled periphery, through which the carrier 5 extends. The support device 18′ is connected by means, which are not illustrated, to the carrier 5, for instance by adhesive, clamping or the like. The periphery of the support device 18′ is so profiled to match the profile of the tubular shaft 1′ that good angular positioning is produced, as is shown in FIG. 8.

1. A urological resectoscope including a tubular shaft (1), through which an optical system (2) and a longitudinally moveable instrument carrier (5) pass, the optical system (2) being supported with respect to the tubular shaft (1) at a spacing from the proximal end of the tubular shaft (1) with a support device (9, 12) and the carrier (5) being positioned with a radial positioning device (10, 15) and a circumferential angular positioning device (11, 12), wherein the support device (12, 18, 18′) is so arranged adjacent to the optical system (2) in the region in which the carrier (5) extends, that it supports the optical system (2) with respect to the tubular shaft (1) with a two point support system and imparts lateral guidance to the carrier (5) in the circumferential angular direction.

2. The resectoscope as claimed in claim (1), wherein the support device (12, 18, 18′) is so constructed that it brings the optical system (2) into engagement with the side of the tubular shaft (1) situated opposite to the support device (12, 18, 19′) in a three point support system.

3. The resectoscope as claimed in claim 1, wherein the support device has two circumferentially spaced, fixed webs (12), which are each arranged in contact with the optical system (2) and the tubular shaft (1) on both sides of the carrier (5) and in sliding contact with it.

4. The resectoscope as claimed in claim 1, wherein the webs (12) are connected to the tubular shaft (1).

5. The resectoscope as claimed in claim 1, including an optical guide tube (4) accommodating the optical system (2), at least in the proximal region of the tubular shaft (1), wherein the webs (12) are connected to the optical guide tube (4).

6. The resectoscope as claimed in claim 1, wherein the webs (12) have guide profiles (15) extending parallel to the tubular shaft (1) for longitudinally moveably guiding the carrier (5) in a radially fixed position.

7. The resectoscope as claimed in claim 1, wherein the carrier (5) has strips (13) contacting the webs (12) in its longitudinal region, with which it is longitudinally moveable on the webs (12).

8. The resectoscope as claimed in claim 1, wherein the support device (18, 18′) is connected to the carrier (5) and is constructed to slide on the optical system (2) and on the tubular shaft (1, 1′).

9. The resectoscope as claimed in claim 8, wherein the tubular shaft (1′) has a non-circular cross section matching the circumferential profile of the support device (18′).
